(19) United States
(12) Patent Application Publication
(10) Pub. No.: US 2023/0052315 A1
(43) Pub. Date: Feb. 16, 2023

(54) VACCINE ADJUVANT COMPRISING AN INVERSE MICROLATEX

(71) Applicant: SOCIÉTÉ D'EXPLOITATION DE PRODUITS POUR LES INDUSTRIES CHIMIQUES-SEPPIC, PARIS CEDEX 07 (FR)

(72) Inventors: Dorothee PLISZCZAK, La Garenne Colombes (FR); Juliette BEN AROUS, La Garenne Colombes (FR); Stéphane MONTEILLET, La Garenne Colombes (FR); David PUJOL, La Garenne Colombes (FR)

(21) Appl. No.: 17/801,224

(22) PCT Filed: Feb. 17, 2021
(86) PCT No.: PCT/EP2021/053873

§ 371 Date: Aug. 19, 2022

(30) Foreign Application Priority Data
Feb. 20, 2020 (FR) 2001698

(51) Int. Cl. A61K 39/39; A61K 39/102; A61K 39/17

(52) U.S. Cl. CPC A61K 39/39; A61K 39/102; A61K 39/17; A61K 2039/55566

(57) ABSTRACT

Disclosed is a vaccine adjuvant including at least one inverse microlatex, the inverse microlatex including at least one oil, at least one surfactant, at least one polymer such as, for example, a polyacrylate that is totally or partially neutralized in the form of alkali metal salts or ammonium salt, the vaccine adjuvant being entirely sterilizable by filtration or by passing through the heat of an autoclave and emulsifiable in one step with the aqueous phase including only a vaccine antigen.

### CROSS-REFERENCE TO RELATED APPLICATIONS

This application is the U.S. national phase of International Application No. PCT/EP2021/053873 filed Feb. 17, 2021 which designated the U.S. and claims priority to French Patent Application No. 2001698 filed Feb. 20, 2020, the entire contents of each of which are hereby incorporated by reference.

### BACKGROUND OF THE INVENTION

### Field of the Invention

The present invention relates to a specific vaccine adjuvant, the preparation thereof and the vaccine comprising same.

### Description of the Related Art

A vaccine composition generally consists of an antigen, an immunogenic compound which induces protection against a disease of interest, and a vaccine adjuvant, which enables the amplification of the immune response of the vaccinated animal against the antigen. The use of adjuvants in vaccine compositions makes it possible in particular to increase the intensity of the humoral or cellular immune response conferred by a dose of vaccine, making it possible to ensure a better level of protection; to prolong the duration of protection conferred by a dose of vaccine; to obtain, with a lower antigen dose, an efficacy equivalent to that conferred by a full dose used without adjuvant; to reduce the number of immunizations needed to ensure vaccine protection.

Immunity adjuvants of various types have been developed in the past. Among the existing technological solutions for obtaining immunity adjuvants, mention may be made of emulsions comprising at least one oily phase and at least one aqueous phase (such as for example Freund's adjuvants), liposomes, synthetic immunostimulatory polymers, adjuvants of biological origin (saponin, chitosan, cytokines, oligonucleotides, etc.) or water-insoluble mineral salts (for example aluminum hydroxide, which is very commonly used).

Oily vaccine adjuvants are composed of oil and surfactants and make it possible to formulate vaccines in the form of emulsions, the aqueous phase of which contains the vaccine antigen. Among the oils used, mention may be made of oils of plant origin, mineral oils, synthetic oils and oils of animal origin. The surfactants present in the oily adjuvants are emulsifying surfactants, having a hydrophilic character characterized by a hydrophilic-lipophilic balance (HLB) value of between 8 and 19, more particularly between 8 and 15. Such a hydrophilic surfactant may consist, for example, of an alkyl polyglycoside or a mixture of alkyl polyglycosides; of saponins; of lecithins; of polyoxyethylated alkanols; of polymers comprising polyoxyethylene and polyoxypropylene blocks; of esters obtained by condensation of a fatty acid, advantageously a fatty acid that is liquid at 20° C., with a sugar polyol, such as for example sorbitol, mannitol or glycerol; of esters obtained by condensation of a fatty acid, advantageously a fatty acid that is liquid at 20° C., with an ethoxylated sugar.

The surfactants present in the oily adjuvants can also be emulsifying surfactants of the “water-in-oil” type, denoting surfactants that have a low enough HLB value, preferably greater than or equal to 1 and less than 8.0, for obtaining water-in-oil emulsions, for which the aqueous phase is dispersed in the lipophilic fatty phase. Among water-in-oil surfactants, mention may be made of anhydrohexitol esters of saturated or unsaturated, linear or branched, aliphatic carboxylic acids comprising from 12 to 22 carbon atoms, optionally substituted with one or more hydroxyl groups, or a mixture of these esters.

The vaccine emulsions obtained can be of water-in-oil, oil-in-water or water-in-oil-in-water type, depending in particular on the nature of the surfactant system used. In particular, adjuvants of the water-in-oil emulsion type make it possible to significantly increase the humoral and cellular response against the vaccine antigen over a prolonged period compared to the non-adjuvanted vaccine or compared to the vaccine adjuvanted with an aqueous adjuvant such as aluminum hydroxide. Such a long-term response can make it possible to reduce the number of vaccine injections. Adjuvants of the water-in-oil emulsion type are notably used for the preparation of vaccine compositions intended for the vaccination of cattle, sheep, goats, fish and avian species against viral, bacterial or parasitic pathogens.

Some synthetic polymers also possess immunostimulatory properties and have been used as vaccine adjuvants.

Among the immunostimulatory polymers used as veterinary vaccine adjuvants, mention may notably be made of block copolymers of polyoxyethylene and polyoxypropylene (POE-POP), polyethyleneimines, acrylic acid homopolymers in the sodium form thereof, copolymers of acrylic acid and acrylic acid esters (also called carbomers). The polymers obtained from acrylic acid, methacrylic acid, acrylic acid esters or methacrylic acid esters can be synthesized according to a precipitation polymerization process in a suitable solvent or by inverse emulsion polymerization, as described in the patent application published under the number FR2922767A1. Among the polymers of acrylic acid, mention will be made, for example, of the polymers sold by the company Lubrizol under the brand name CARBOPOL™ described in particular in the US patents published under the numbers U.S. Pat. Nos. 5,373,044, 2,798,053 and in European patent application EP 0301532A2.

Carbomers (or acrylic acid polymers) are used at weight contents of the order of one percent for applications as a vaccine adjuvant, and the dilution thereof is in the form of an easily injectable, fluid and translucent vaccine.

These polymeric adjuvants have very good safety and induce a strong short-term response against the associated antigen, and are notably used for the vaccination of pigs, as described for example in the patent application published under the number WO2007094893).

A promising avenue of research consists in formulating these polymeric adjuvants in combination with emulsion-type oily adjuvants, with the aim of combining the immunostimulatory properties of the two types of adjuvants to obtain an adjuvant of improved performance, as described for example in the US patent published under the number U.S. Pat. No. 3,919,411.

However, the combination of these two technologies (i.e. oily adjuvants and polyacrylate gels) in order to obtain a ready-to-use polymeric oily immunity adjuvant that contains a polyacrylate, is stable and can be directly emulsified by the user is a galenic challenge, notably with regard to the stability and sterilization of the adjuvant.

For the purposes of the present invention, a “ready-to-use polymeric oily immunity adjuvant” is understood to mean a mixture consisting of an oily phase containing at least one surfactant and one polymer, which mixture is already sterilized and can immediately be used by mixing with the aqueous antigen medium in an emulsification step. When said mixture comes into contact with an aqueous phase (containing an antigen and/or an active principle), an emulsion is formed owing to the use of a low- or high-shear stirring system. This type of adjuvant (hereinafter referred to as “polymeric oily adjuvant”) is used with the aim of obtaining prophylactic or therapeutic vaccine emulsions which are stable over time.

Several strategies can be considered and are identified in order to prepare a polymeric oily adjuvant, but each one poses some technical problems:
1) The first approach consists in adding to an oily phase at least one polyacrylic acid, a polymer in which the carboxylic groups that are not salified, and which is in the form of a powder. In this case, the powder remains difficult to stabilize in the oil in the form of a suspension and may exhibit sedimentation problems over time. Moreover, the adjuvant obtained in this way leads to the production of emulsions of acid nature, since the polymer is not neutralized and more particularly cannot be neutralized during the emulsification process. All these parameters mean that this technical solution is unsatisfactory.
2) The second approach consists in adding an aqueous gel, formed beforehand by adding at least one polyacrylic acid to water, to an oily adjuvant. In this case, the dispersion of an aqueous gel in an oily phase has the first constraint of not guaranteeing the homogeneity of the mixture obtained at the end of this process. In addition, this approach has the major risk of leading to a phase separation of the dispersed phase, then leading to the non-homogeneity of the desired product.
3) The third approach is to disperse a polymeric adjuvant such as a polyacrylate, that is in the form of an inverse latex (or W/O emulsion, the dispersed aqueous phase of which comprises a polyacrylate, the carboxylic functions of which have been previously neutralized in the form of an alkali metal salt or an ammonium salt) in the oily adjuvant. However, a phase separation is observed over time, leading to heterogeneity of the oily adjuvant.

The difficulties in formulating a ready-to-use polymeric oily adjuvant are also linked to the steps of sterilizing each compound that is incorporated into the composition of the vaccine. Specifically, vaccine compositions dedicated to administration by injection must be sterilized before formulating with the antigen under aseptic conditions. Among the sterilization methods that can be used, note can be made of heat sterilization of the product in an autoclave, followed by a step of filtration sterilization on a filter with a pore diameter of 0.2 micrometers or a step of gamma-ray irradiation.

Since the vaccine compositions are in the form of an emulsion that cannot be sterilized, it is therefore necessary to sterilize the oily adjuvants, by filtration or by heat by passing through an autoclave, before the step of emulsifying with the antigen medium. It should also be noted that the surfactants contained in the oily adjuvants are generally not compatible with irradiation sterilization.

As regards the present polymeric vaccine adjuvants on the market, the crosslinked structures thereof and the thickening properties thereof make both filtration operations on sterilizing filters with a pore diameter of 0.2 micrometers, and irradiation sterilization operations, impossible. Therefore, exposure to heat by passing through an autoclave is the only sterilization technique suitable for polymeric vaccine adjuvants. Since this technique requires the preparation of a dilute solution of polymeric adjuvant in water, said polymeric adjuvants cannot be sterilized by this route when they are combined with oily adjuvants.

It follows from the elements indicated above that to prepare a sterile vaccine, containing a combination of a polymeric adjuvant and an oily adjuvant, it is necessary for:
the oily adjuvant to be sterilized by heating in an autoclave or by sterilizing filtration on the one hand, and
the polymeric adjuvant to be hydrated, diluted and sterilized in solution by heating in an autoclave, on the other hand, and
the polymeric adjuvant to be mixed aseptically with the aqueous antigen medium, and
the aqueous mixture of the polymeric adjuvant and the antigen medium to be emulsified aseptically with the sterile oily phase.

This method is therefore considered by those skilled in the art to be expensive since it comprises many steps, consumes energy, and does not allow direct marketing of the mixture of the combination of the polymeric adjuvant and the oily adjuvant.

Thus, there is a need for a solution which consists in providing a polymeric oily adjuvant containing at least one oil and at least one polymer such as for example a polyacrylate, said polymeric oily adjuvant being stable over time for at least 1 year and more. particularly at least 2 years at 20° C. (“stable” is understood to mean the absence of phase separation, of solidification of the polymer during storage), easily sterilizable and which makes it possible to achieve emulsions that are stable over time at +4° C. for 1 year and for at least 1 month at +37° C. (i.e. exhibiting neither sedimentation or phase separation). The polymeric oily adjuvant according to the invention must make it possible to obtain vaccine compositions that are effective from an immunological point of view.

### SUMMARY OF THE INVENTION

A solution of the present invention is a vaccine adjuvant comprising at least one inverse microlatex.

For the purposes of the present invention, an inverse microlatex denotes an inverse microemulsion comprising at least one polyelectrolyte-type polymer.

For the purposes of the present invention, a “microemulsion” denotes a mixture of two immiscible liquids which is thermodynamically stable, stabilized by the presence of a surfactant system comprising at least one emulsifying surfactant. A microemulsion is generally transparent because the size of the droplets of the dispersed phase is characterized by a mean particle diameter of less than or equal to 200 nanometers, and preferably less than or equal to 100 nanometers.

For the purposes of the present invention, an “inverse microemulsion” denotes a microemulsion as defined above, for which the dispersed phase is an aqueous phase and the continuous phase is an oily phase.

For the purposes of the present invention, a “polyelectrolyte-type polymer” denotes a polymer in which all or some of the monomer units present in the polymer bear an ionized chemical function. Thus, an anionic polyelectrolyte-type polymer predominantly comprises monomer units possessing an anionic function and a cationic polyelectrolyte-type polymer predominantly comprises monomer units possessing a cationic function.

For the purposes of the present invention, an “anionic and crosslinked polyelectrolyte-type polymer” denotes an anionic polyelectrolyte-type polymer as defined above and which comprises, via its constituent monomer units, at least one monomer unit possessing at least two reactive functions that may be employed during the polymerization reaction and that thus make it possible to link together at least two polymer chains.

Inverse microlatexes are prepared by implementing a process which comprises the following steps:
a step a) of preparing an aqueous solution containing the monomers and the optional various additives (such as for example a crosslinking monomer),
a step b) of adding, to the aqueous phase obtained in step a), at least one oil, at least one surfactant and of mixing these various components,
a step c) of adding a radical initiator to initiate a radical polymerization reaction in an adiabatic medium, and
a step d) of homogenizing with mechanical stirring of the reaction medium obtained during step c).

Such a process for preparing an inverse microlatex is described in European patent application published under the number EP 1 371 692 A1, which is incorporated by reference in the present patent application.

Depending on the case, the vaccine adjuvant according to the invention may have one or more of the following characteristics:
the inverse microlatex comprises an oily phase, an aqueous phase, at least one water-in-oil (W/O) surfactant, at least one oil-in-water (O/W) surfactant and an anionic and crosslinked polyelectrolyte; with said anionic and crosslinked polyelectrolyte comprising at least one crosslinking monomer and at least one hydrophilic monomer unit;
the hydrophilic monomer unit originates from acrylic acid that is completely or partially salified with an alkali or alkaline-earth metal salt or an ammonium salt;
the acrylic acid is completely or partially salified with a sodium salt or an ammonium salt, preferably with a sodium salt;
the anionic and crosslinked polyelectrolyte comprises a monomer unit of formula (1):

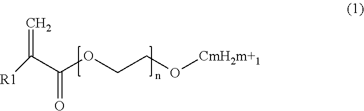

with: R1 chosen from —H, —CH₃, —C₂H₅ and —C₃H₇, preferably —CH₃, n between 0 and 50, and m between 8 and 22;
said adjuvant further comprises an oil (H₁), at least one water-in-oil surfactant (E₁) and at least one oil-in-water surfactant (E₂);
the adjuvant comprises between 1% and 10% by weight of water-in-oil surfactant (E₁), preferably from 3% to 8% by weight;
the adjuvant comprises between 1% and 10% by weight of water-in-oil surfactant (E₂), preferably from 3% to 8% by weight;
the adjuvant comprises, per 100% of its weight:
a) from 50% to 97.5% by weight of said oil (H₁), preferably from 60% to 90%;
b) from 1% to 10% by weight of said water-in-oil surfactant (E₁), preferably from 3% to 8%;
c) from 1% to 10% by weight of said oil-in-water surfactant (E₂), preferably from 3% to 8%; and
d) from 0.5% to 30% by weight of at least one inverse microlatex, preferably from 1 to 10%, more preferentially between 1% and 10%,
   it being understood that the sum of the weight contents a)+b)+c)+d) is equal to 100%.
the vaccine adjuvant according to the invention, characterized in that the oil (H₁) is a white mineral oil. It is noted that the oil (H₁) could also be a mineral oil, such as for example liquid paraffin, liquid petroleum jelly, or an isoparaffin.

Preferentially, the inverse microlatex included in the vaccine adjuvant according to the invention will comprise, per 100% of its weight:
a′) from 10% to 40% by weight of water, preferably from 12% to 30% by weight,
b′) from 30% to 50% by weight of oil (H₂), preferably from 38% to 50% by weight,
c′) from 5% to 30% by weight, preferably from 10% to 25% by weight of a mixture of at least one water-in-oil surfactant (E′₁) and of at least one oil-in-water surfactant (E′₂),
d′) from 5% to 35% by weight, preferably from 10% to 30% by weight of said anionic and crosslinked polyelectrolyte,

it being understood that the sum of the weight contents a′)+b′)+c′)+d′) is equal to 100%.

The oil (H₁) included in the vaccine adjuvant that is the subject of the present invention is identical to or different from the oil (H₂) included in the inverse microlatex.

According to one particular aspect, the oil (H₁) included in the vaccine adjuvant that is the subject of the present invention is identical to the oil (H₂) included in the inverse microlatex. The oils (H₂) and the oils (H₁) are chosen in particular from:
oils of plant origin, such as sweet almond oil, coconut oil, monoi oil, castor oil, jojoba oil, olive oil, rapeseed oil, peanut oil, sunflower oil, wheat germ oil, corn germ oil, soybean oil, cottonseed oil, alfalfa oil, poppy oil, red kuri squash oil, evening primrose oil, millet oil, barley oil, rye oil, safflower oil, candlenut oil, passionflower oil, hazelnut oil, palm oil, shea butter, apricot kernel oil, calophyllum oil, sisymbrium oil, avocado oil, calendula oil;
plant oils and the ethoxylated methyl esters thereof;
oils of animal origin, such as squalene or squalane;
synthetic oils, notably fatty acid esters such as butyl myristate, propyl myristate, cetyl myristate, isopropyl palmitate, butyl stearate, hexadecyl stea rate, isopropyl stea rate, isocetyl stearate, dodecyl oleate, hexyl laurate, propylene glycol dicaprylate, esters derived from lanolic acid, such as isopropyl lanolate, isocetyl lanolate, fatty acid monoglycerides, diglycerides and triglycerides such as glycerol triheptanoate, alkylbenzoates, poly(alpha-olefin)s, polyolefins such as poly(isobutene), synthetic isoalkanes such as isohexadecane, isododecane, and perfluorinated oils. Silicone oils are also capable of being used in the context of the present invention.
Among the latter, mention may more particularly be made of polydimethylsiloxanes, polymethylphenylsiloxanes, silicones modified by amines, silicones modified by fatty acids, silicones modified by alcohols, silicones modified by alcohols and fatty acids, silicones modified by polyether groups, epoxy-modified silicones, silicones modified by fluorinated groups, cyclic silicones and silicones modified by alkyl groups. However, for practical reasons, it may be desirable for the fatty phase not to include silicone oil;
mineral oils, hydrocarbons, such as liquid paraffin, liquid petroleum jelly, white mineral oils and isoparaffins, obtained by distillation of petroleum and by the implementation of subsequent treatment steps such as desulfurization, deasphalting, extraction of aromatic compounds, extraction of waxes and other finishing treatment steps. White oil denotes mineral oils that comply with the FDA 21 CFR 172.878 and CFR 178.3620(a) regulations, listed in the US Pharmacopeia, US XXIII (1995) and that comply with the purity requirements of the European Pharmacopoeia (2008). Mention may be made, for example, of the oils sold under the brand names Marcol™, Primol™, Drakeol™, Eolane™, Klearol™, Puretol™;
light oils. For the purposes of the present invention, a “light oil” denotes an oil (H₂), with a low boiling point (from 100° C. to 250° C. at atmospheric pressure), included in the fatty phase of the inverse microlatex, also consisting of at least one oil with a higher boiling point; said light oil being intended to be evaporated during a step of concentration by distillation of the inverse microlatex formed in order to obtain a concentrated inverse microlatex. As a light oil that meets this definition, mention may be made of isoparaffins comprising from 7 to 14 carbon atoms sold under the brand names Isopar™C, Isopar™E, Isopar™G, Isopar™H, Isopar™L and Isopar™M.

The water-in-oil surfactant (E₁) included in the vaccine adjuvant that is the subject of the present invention is identical to or different from the water-in-oil surfactant (E′₁) included in the inverse microlatex.

According to one particular aspect, the water-in-oil surfactant (E₁) included in the vaccine adjuvant that is the subject of the present invention is identical to the water-in-oil surfactant (E′₁) included in the inverse microlatex.

For the purposes of the present invention, a “surfactant” denotes a compound which modifies the surface tension between two surfaces and which is an amphiphilic molecule, that is to say that it has in its structure a lipophilic part and another hydrophilic part. Thus, a surfactant makes it possible to solubilize and/or disperse a phase of a certain polarity in another phase of different polarity.

The term “water-in-oil surfactant” denotes surfactants that have a low enough HLB value, preferably greater than or equal to 1 and less than 8.0, for obtaining water-in-oil emulsions, for which the aqueous phase is dispersed in the lipophilic fatty phase.

Among the water-in-oil surfactants (E₁) and (E′₁), mention may be made of anhydrohexitol esters of saturated or unsaturated, linear or branched, aliphatic carboxylic acids comprising from 12 to 22 carbon atoms, optionally substituted with one or more hydroxyl groups, or a mixture of these esters.

The term “hexitol” denotes hexols derived from hexoses such as sorbitol, mannitol, dulcitol (also known as galactitol) or iditol.

The term “anhydrohexitol” denotes the products resulting from the dehydration of hexitols. Examples of anhydrohexitols include, for example, anhydrosorbitols, anhydromannitols, anhydrodulcitols or anhydroiditols. The term “anhydrohexitols” denotes monoanhydrohexitols (such as for example sorbitan, mannitan, dulcitan, iditan), optionally as a mixture with dianhydrohexitols (such as for example isosorbide, isomannide, isodulcide, isoidide) obtained as by-products during the same dehydration reaction.

The term “mixture of esters” denotes the esters obtained either from a single acid and from a single hexitol, or from a single acid and from a mixture of several hexitols, or from a mixture of several acids and from a single hexitol, or from a mixture of several acids with several hexitols.

The expression “anhydrohexitol ester of saturated or unsaturated, linear or branched, aliphatic carboxylic acids comprising from 12 to 22 carbon atoms, optionally substituted with one or more hydroxyl groups” denotes for example the esters of acids chosen from dodecanoic acids, dodecenoic acids, tetradecanoic acids, tetradecenoic acids, hexadecanoic acids, hexadecenoic acids, octadecanoic acids, octadecenoic acids, octadecadienoic acids, octadecatrienoic acids, octadecatetraenoic acids, eicosanoic acids, eicosenoic acids, eicosadienoic acids, docosanoic acids, docosenoic acids, hydroxyhexadecanoic acids, hydroxyoctadecanoic acids, dihydroxydocosanoic acids or dihydroxyoctadecanoic acids.

The expression “anhydrohexitol ester of saturated or unsaturated, linear or branched, aliphatic carboxylic acids comprising from 12 to 22 carbon atoms, optionally substituted with one or more hydroxyl groups” denotes for example the esters of acids chosen from lauric acid, isolauric acid, 4-dodecenoic acid, 5-dodecenoic acid, myristic acid, palmitic acid, hypogeic acid, stearic acid, isostearic acid, oleic acid, isooleic acid, linoleic acid, isogeranic acid, linolenic acid, arachidic acid, 10,13-eicosadienoic acid, behenic acid, erucidic acid, cetoleic acid, brassic acid, 3-hydroxyhexadecanoic acid, 4-hydroxyhexadecanoic acid, 11-hydroxyhexadecanoic acid, 16-hydroxyhexadecanoic acid, 12-hydroxystearic acid, brasileic acid or 8,9-dihydroxystearic acid.

These esters are obtained by esterification of the corresponding acids and anhydrohexitols. The esterification reaction is known to those skilled in the art; it is described in numerous patents and reference books.

Among the anhydrohexitol esters of saturated or unsaturated, linear or branched, aliphatic carboxylic acids comprising from 12 to 22 carbon atoms, optionally substituted with one or more hydroxyl groups, mention may be made of sorbitan laurate (sold under the brand name Montane™20), mannitan laurate, dulcitan laurate, sorbitan isolaurate, mannitan isolaurate, ducitan isolaurate, sorbitan palmitate, mannitan palmitate, dulcitan palmitate, sorbitan stearate (sold under the brand name Montane™60), mannitan stearate, dulcitan stearate, sorbitan isostearate (sold under the brand name Montane™70), mannitan isostearate, dulcitan isostearate, sorbitan oleate (sold under the brand name Montane™80), mannitan oleate (sold under the brand name Montanide™80), dulcitan oleate, sorbitan sesquioleate (sold under the brand name Montane™83), mannitan sesquioleate, sorbitan trioleate (sold under the brand name Montane™85), mannitan trioleate, sorbitan benenate, mannitan behenate, sorbitan arachinate, and mannitan arachinate.

Among the water-in-oil surfactants (E₁) and (E′1), mention may also be made of sorbitan oleate ethoxylated with 5 mol of ethylene oxide (5 EO) sold by the applicant under the name Montanox™ 81, diethoxylated (2 EO) oleocetyl alcohol sold by the applicant under the name Simulsol™ 0072.

The term “oil-in-water surfactant” (E₂) and (E′₂) denotes surfactants that have a high enough HLB value, preferably greater than or equal to 8.0 and less than or equal to 20, preferably greater than or equal to 8.0 and less than or equal to 15.0, for obtaining oil-in-water emulsions, for which the lipophilic fatty phase is dispersed in the aqueous phase.

Among the oil-in-water surfactants (E₂) and (E′₂), mention may be made of anhydrohexitol esters of saturated or unsaturated, linear or branched, aliphatic carboxylic acids comprising from 12 to 22 carbon atoms, optionally substituted with one or more hydroxyl groups, or a mixture of these esters, which are subsequently subjected to a step of adding ethylene oxide to a variable degree of between 2 and 30 molar equivalents of ethylene oxide.

Among the anhydrohexitol esters of saturated or unsaturated, linear or branched, aliphatic carboxylic acids comprising from 12 to 22 carbon atoms, optionally substituted with one or more hydroxyl groups, and ethoxylated, mention may in particular be made of ethoxylated sorbitan esters, and more particularly sorbitan oleate ethoxylated with 20 mol of ethylene oxide (20 EO), sold by the applicant under the name Montanox™ 80, ethoxylated sorbitan oleate containing 15 moles of ethylene oxide (15 EO), sorbitan oleate ethoxylated with 10 mol of ethylene oxide (10 EO), sorbitan oleate ethoxylated with 5 mol of ethylene oxide (5 EO), mannitan oleate ethoxylated with 20 mol of ethylene oxide (20 EO), mannitan oleate ethoxylated with 15 mol of ethylene oxide (15 EO), mannitan oleate ethoxylated with 10 mol of ethylene oxide (10 EO), mannitan oleate ethoxylated with 5 mol of ethylene oxide (5 EO), sorbitan stearate ethoxylated with 20 mol of ethylene oxide (20 EO), sorbitan stearate ethoxylated with 10 mol of ethylene oxide (10 EO), sorbitan stearate ethoxylated with 5 mol of ethylene oxide (5 EO), mannitan stearate ethoxylated with 20 mol of ethylene oxide (20 EO), mannitan stearate ethoxylated with 10 mol of ethylene oxide (10 EO), mannitan stearate ethoxylated with 5 mol of ethylene oxide (5 EO), sorbitan laurate ethoxylated with 20 mol of ethylene oxide (20 EO) sold by the applicant under the name Montanox™ 20, sorbitan laurate ethoxylated with 10 mol of ethylene oxide (10 EO), sorbitan laurate ethoxylated with 5 mol of ethylene oxide (5 EO), mannitan laurate ethoxylated with 20 mol of ethylene oxide (20 EO), mannitan laurate ethoxylated with 10 mol of ethylene oxide (10 EO), mannitan laurate ethoxylated with 5 mol of ethylene oxide (5 EO), sorbitan trioleate ethoxylated with 5 mol of ethylene oxide, sorbitan trioleate ethoxylated with 10 mol of ethylene oxide, sorbitan trioleate ethoxylated with 20 mol of ethylene oxide, sorbitan trioleate ethoxylated with 25 mol of ethylene oxide sold by the applicant under the name Montanox™ 85, mannitan trioleate ethoxylated with 5 mol of ethylene oxide, mannitan trioleate ethoxylated with 10 mol of ethylene oxide, mannitan trioleate ethoxylated with 20 mol of ethylene oxide, mannitan trioleate ethoxylated with 25 mol of ethylene oxide.

Among the oil-in-water surfactants (E₂) and (E′₂), mention may be made of ethoxylated plant oils such as, for example, castor oil ethoxylated with 25 EO sold by the applicant under the name Simulsol™ 1292, castor oil ethoxylated with 40 EO sold by the applicant under the name Simulsol™ OL 50, corn oil ethoxylated with 3 mol of ethylene oxide (3 EO), corn oil ethoxylated with 8 mol of ethylene oxide (8 EO), corn oil ethoxylated with 10 mol of ethylene oxide (10 EO), corn oil ethoxylated with 20 mol of ethylene oxide (20 EO), corn oil ethoxylated with 30 mol of ethylene oxide (30 EO), corn oil ethoxylated with 40 mol of ethylene oxide (40 EO), rapeseed oil ethoxylated with 3 mol of ethylene oxide (3 EO), rapeseed oil ethoxylated with 10 mol of ethylene oxide (10 EO), rapeseed oil ethoxylated with 20 mol of ethylene oxide (20 EO), rapeseed oil ethoxylated with 30 mol of ethylene oxide (30 EO), rapeseed oil ethoxylated with 40 mol of ethylene oxide (40 EO), sunflower oil ethoxylated with 3 mol of ethylene oxide (3 EO), sunflower oil ethoxylated with 10 mol of ethylene oxide (10 EO), sunflower oil ethoxylated with 20 mol of ethylene oxide (20 EO), sunflower oil ethoxylated with 30 mol of ethylene oxide (30 EO), sunflower oil ethoxylated with 40 mol of ethylene oxide (40 EO).

Among the oil-in-water surfactants (E₂) and (E′₂), mention may be made of alkyl polyglycosides, more particularly alkyl polyglucosides and alkyl polyxylosides, or a mixture of alkyl glycosides, lecithins, saponins, polyoxyethylated alkanols, polymers comprising polyoxyethylene and polyoxypropylene blocks, lauryl alcohol ethoxylated with 7 mol of ethylene oxide (7 EO) sold by the applicant under the name Silmulsol™ P7, pentaethoxylated (5 EO) oleocetyl alcohol, octaethoxylated (8 EO) oleocetyl alcohol, decaethoxylated (10 EO) oleocetyl alcohol sold by the applicant under the name Simulsol™ OC 710, or polyethoxylated sorbitan hexaoleates sold under the names G-1086™ and G1096™.

A crosslinking monomer unit denotes a unit derived from a monomer possessing at least two reactive functions by which covalent bonds can be established between the extending polymer chains and said crosslinking monomer. For example, a crosslinking monomer unit may be derived from a monomer which may comprise at least two ethylenic functions in its structure and, when engaged in a radical polymerization reaction with acrylic monomers, said crosslinking monomer can bond covalently to two chains of acrylic polymer during the propagation step, to obtain a crosslinked polymer.

A crosslinked polymer denotes a nonlinear polymer that is in the form of a three-dimensional network which is insoluble in water but which can swell in water and which therefore results in a chemical gel being obtained.

A crosslinked polymer denotes a polymer composed of at least one crosslinking monomer unit and of at least one other monomer unit, and more particularly of a hydrophilic monomer unit. For the purposes of the present invention, a hydrophilic monomer unit is understood to mean a monomer unit resulting from a monomer that is soluble in water and more particularly that is soluble in water at a temperature above or equal to 5° C., more particularly at a temperature above or equal to 10° C., more particularly at a temperature above or equal to 20° C.

The crosslinked polymer may comprise hydrophilic monomer units derived from: 2-methyl-2-[(1-oxo-2-propenyl)amino]-1-propanesulfonic acid in free acid form or partially or totally salified form; acrylic acid in free acid form or partially or totally salified form, methacrylic acid in free acid form or partially or totally salified form, itaconic acid in free acid form or partially or totally salified form, 2-carboxyethyl acrylic acid in free acid form or partially or totally salified form, maleic acid in free acid form or partially or totally salified form, acrylamide, N,N-dimethylacrylamide, methacrylamide, N-isopropylacrylamide, 2-hydroxyethyl acrylate, 2,3-dihydroxypropyl acrylate, 2-hydroxyethyl methacrylate, 2,3-dihydroxypropyl methacrylate, vinyl pyrrolidone.

A crosslinking monomer unit denotes a monomer unit derived from a diethylenic or polyethylenic monomer, notably chosen from ethylene glycol dimethacrylate, diethylene glycol diacrylate, ethylene glycol diacrylate, diallylurea, triallylamine, trimethylolpropane triacrylate, methylenebis(acrylamide) or a mixture of these compounds, diallyloxyacetic acid or a salt thereof such as sodium diallyloxyacetate, or a mixture of these compounds.

The surfactants present in the adjuvant according to the invention are water-in-oil surfactants (E₁) or water-in-oil surfactants (E′1), as defined and described above, having a lipophilic nature characterized by an HLB value greater than or equal to 1 and less than 8 and oil-in-water surfactants (E₂) or oil-in-water surfactants (E′₂), as defined and described above, having a hydrophilic nature characterized by an HLB value greater than or equal to 8 and less than or equal to 20.

Another subject of the present invention is the use of an inverse microlatex as defined previously, for the preparation of a vaccine adjuvant.

The process for preparing a vaccine adjuvant according to the invention comprises a step of sterilizing the vaccine adjuvant by sterilizing filtration or autoclaving.

The filtration will preferably be carried out on a filter having pores with a mean diameter of less than or equal to 0.22 microns (see standard ISO 13408-2:2018(en)).

Before being filtered, the adjuvant may be prefiltered. By way of example, the adjuvant can be prefiltered using hydrophobic filters having pores with a mean diameter of 0.45 μm. The prefiltration and filtration steps can be carried out in a single step, which involves the use of double-membrane hydrophobic filter, of which a first membrane has pores with a mean diameter of 0.45 μm and the second membrane has pores with a mean diameter of 0.2 μm, or the combination of a first hydrophobic filter having pores with a mean diameter of 0.45 μm and a second hydrophobic filter having pores with a mean diameter of 0.2 μm. This means that the first membrane or the first filter has larger pores than the second membrane or the second filter. Ideally, the first membrane or the first filter have pores with a diameter greater than or equal to 0.3 μm, preferably pores with a diameter less than or equal to 0.6 μm and more particularly equal to 0.45 μm. The second membrane has pores with a diameter less than or equal to 0.22 μm in order to obtain a sterilizing action.

The filters and the membranes used for the filtration and/or the prefiltration of the adjuvant can consist of polymeric supports of PTFE (polytetrafluoroethylene) or PP (polypropylene) type.

Preferentially, said process for preparing an adjuvant according to the invention comprises the following steps:
a) preparing, with mechanical stirring and at ambient temperature, an oily phase comprising at least one oil and an emulsifying system comprising at least one water-in-oil surfactant (E₁) and/or oil-in-water surfactant (E₂);
b) adding the at least one inverse microlatex with mechanical stirring at ambient temperature;
c) maintaining the mechanical stirring, at ambient temperature, until a homogeneous mixture is obtained.

For the purposes of the present invention, ambient temperature is understood to mean a temperature above or equal to 15° C. and below or equal to 30° C.

Another subject of the present invention is a vaccine comprising the adjuvant according to the invention and also at least one aqueous solution (S) of at least one antigen or of at least one in vivo generator of a compound comprising an amino acid sequence.

Preferably the vaccine will comprise:
from 10% to 80% by weight of the adjuvant according to the invention, and
from 20% to 90% by weight of the aqueous solution (S).

Preferably, said vaccine is in the form of a water-in-oil emulsion or an oil-in-water emulsion. An antigen or at least one in vivo generator of a compound comprising an amino acid sequence denotes either killed microorganisms, such as viruses, bacteria or parasites, or purified fractions of these microorganisms, or living microorganisms, the pathogenicity of which has been reduced. Examples of viruses which can constitute an antigen according to the present invention include orthomyxoviruses such as the influenza virus, paramyxoviruses such as the Newcastle disease virus, coronaviruses such as the infectious bronchitis virus, herpes viruses such as Aujeszky's disease virus or Marek's disease virus. As a microorganism of bacterial type which can constitute an antigen according to the present invention, mention may be made E. coli, and those of the Pasteurella, Avibacterium, Staphylococcus and Streptococcus genera. Examples of parasites include those of the Eimeria, Trypanosoma, and Leishmania genera. Mention may also be made of recombinant viruses, in particular nonenveloped viruses, such as adenoviruses, the vaccinia virus, the canarypox virus, herpes viruses or baculoviruses. Also denoted is a live, nonenveloped, recombinant viral vector, the genome of which contains, preferably inserted into a part that is not essential for replication of the corresponding enveloped virus, a sequence encoding an antigen subunit which induces antibody synthesis and/or a protective effect against the abovementioned enveloped virus or pathogenic microorganism; these antigen subunits may, for example, be a protein, a glycoprotein, a peptide or a fraction which is a peptide fraction and/or protective against an infection with a living microorganism such as an enveloped virus, a bacterium or a parasite. The exogenous gene inserted into the microorganism may, for example, be derived from an Aujeszky virus. Mention may in particular be made of a recombinant plasmid made up of a nucleotide sequence, into which an exogenous nucleotide sequence, originating from a pathogenic microorganism or virus, is inserted. The aim of the latter nucleotide sequence is to allow the expression of a compound comprising an amino acid sequence, the aim of this compound itself being to trigger an immune reaction in a host organism.

The vaccine as defined above comprises an antigen concentration which depends on the nature of this antigen and on the nature of the individual treated. It is, however, particularly noteworthy that an adjuvant according to the invention makes it possible to significantly decrease the usual antigen dose required. The suitable antigen concentration can be determined conventionally by those skilled in the art. Generally, this dose is about 0.1 [symbol] g/cm³ to 1 g/cm³, more generally between 1 [symbol] g/cm³ and 100 mg/cm³. The concentration of said in vivo generator in the composition according to the invention depends, here again, in particular on the nature of said generator and on the host to which it is administered. This concentration can be readily determined by those skilled in the art, on the basis of routine experiment. By way of indication, when the in vivo generator is a recombinant microorganism, its concentration in the composition according to the invention is in general between 10² and 10¹⁵ microorganisms/cm³ and preferably between 10⁵ and 10¹² microorganisms/cm³. When the in vivo generator is a recombinant plasmid, its concentration in the composition obtained according to the process which is the subject of the invention can be between 0.01 g/dm³ and 100 g/dm³. The vaccine, as defined above, is prepared by mixing the adjuvant phase and the antigen phase, optionally adding water or a pharmaceutically acceptable diluent medium. The process for preparing the vaccine according to the invention comprises the following steps:
a) preparing the vaccine adjuvant according to the invention,
b) mixing the vaccine adjuvant obtained in step a) with an antigen medium.

Preferably the antigen medium is intended to form a vaccine, reference will be made to a vaccine antigen medium.

An antigen medium denotes an aqueous medium comprising at least one antigen or at least one in vivo generator of a compound comprising an amino acid sequence, as described above. Preferably the mixture will be such that the vaccine will comprise, per 100% of its weight, between 10% and 80% by weight of adjuvant and between 20% and 90% by weight of antigen medium, preferably between 50% and 80% by weight of adjuvant and between 20% and 50% by weight of antigen medium, and even more preferably between 50% and 70% by weight of adjuvant and between 30% and 50% by weight of antigen medium.

During step b), an immunostimulant chosen from saponins, animal and/or plant and/or mineral and/or synthetic oils, surfactants, aluminum hydroxide, lecithins and lecithin derivatives may optionally be added to the mixture.

Preferentially, in step b) the antigen medium will be added to the adjuvant gradually with high-shear stirring to form an emulsion.

At the end of the emulsification process, a vaccine in the form of a stable and homogeneous emulsion, preferably of water-in-oil type, is obtained.

The final vaccine can be administered as soon as it is manufactured and can be stored for at least 1 year, at a temperature of +4° C. (depending on the nature of the antigen(s) present in the vaccine and their physicochemical stability over time).

The vaccine is intended to be administered in human or veterinary therapy by injection and by oral, parenteral, mucosal or in ovo routes.

### BRIEF DESCRIPTION OF THE DRAWINGS

FIG. 1 is a graph representing the response of the IgG1 antibody against the OVA antigen in mice for a vaccine comprising the ADJ3 adjuvant according to the invention.

FIG. 2 is a graph representing the response of the IgG2a antibody against the OVA antigen in mice for a vaccine comprising the ADJ3 adjuvant according to the invention.

FIG. 3 is a graph representing the change in the titre of antibodies against Newcastle disease LaSota strain (NDV) from DO to D28.

FIG. 4 is a graph representing the change in the titre of antibodies against H9N2 avian influenza (AIV) from DO to D28.

FIG. 5 is a graph representing the degree of protection (number of animals protected/total number) obtained by the test vaccine group in comparison with the control group.

### DESCRIPTION OF THE PREFERRED EMBODIMENTS

Examples of adjuvants according to the invention are presented below.

### Example 1: Preparation of Inverse Microlatexes Based on Sodium Polyacrylate

1.1 Preparation of Inverse Microlatexes (A), (B), (C) and (D)

Inverse microlatexes comprising, as polymer, a crosslinked sodium polyacrylate, are prepared according to the teachings of the European patent published under the number 1 371 692 B1, which is incorporated here by reference. More particularly the teachings of paragraph [0021], paragraphs [0025] and [0026], paragraphs [0033] to [0048], and even more particularly paragraphs [0039] to [0041] (example 2) of the European patent published under the number 1 371 692 B1, are used to prepare the inverse microlatexes. For each of the microlatexes prepared, a fluid white mineral oil, Marcol™52 sold by the company Exxon Mobil, is used. The same process for preparing inverse microlatexes is carried out in the presence of various weight concentrations of surfactants, and makes it possible to obtain:
the inverse microlatex (A) when the weight amount of surfactants is equal to 14%,
the inverse microlatex (B) when the weight amount of surfactants is equal to 18%,
the inverse microlatex (C) when the weight amount of surfactants is equal to 22%,
the inverse microlatex (D) when the weight amount of surfactants is equal to 25%.

The inverse microlatexes (A), (B), (C) and (D), obtained after radical polymerization, are in the form of opalescent to translucent oily compositions. These inverse microlatexes contain 60% by weight of a mixture of oily phase and surfactants, 15% by weight of crosslinked sodium polyacrylate and 25% by weight of water.

1.2 Preparation of Inverse Microlatexes (E), (F), (G) and (H)

The process for preparing the inverse microlatex (A) is carried out using mannitan oleate as water-in-oil surfactant instead of sorbitan oleate in order to obtain the inverse microlatex (E).

The process for preparing the inverse microlatex (B) is carried out using mannitan oleate as water-in-oil surfactant instead of sorbitan oleate in order to obtain the inverse microlatex (F).

The process for preparing the inverse microlatex (C) is carried out using mannitan oleate as water-in-oil surfactant instead of sorbitan oleate in order to obtain the inverse microlatex (G).

The process for preparing the inverse microlatex (D) is carried out using mannitan oleate as water-in-oil surfactant instead of sorbitan oleate in order to obtain the inverse microlatex (H).

The inverse microlatexes (E), (F), (G) and (H) are in the form of an opalescent to translucent oily composition, containing 60% by weight of a mixture of oily phase and surfactants, 15% by weight of crosslinked sodium polyacrylate and 25% by weight of water.

### Example 2: 1.1 Preparation of Inverse Microlatexes (A′), (B′), (C′) and (D′)

The process for preparing the inverse microlatexes (A), (B), (C), and (D), described in example 1.1, is carried out in the presence of a mixture of acrylic acid and tetraethoxylated lauroyl methacrylate (2 mol %), in order to obtain respectively the inverse microlatexes (A′), (B′), (C′), and (D′).

The inverse microlatexes (A′), (B′), (C′), and (D′) are in the form of opalescent to translucent oily compositions, containing 60% by weight of a mixture of oily phase and surfactants, 15% by weight of crosslinked copolymer of acrylic acid and tetraethoxylated lauroyl methacrylate and 25% by weight of water.

### Example 3: Preparation of Inverse Microlatexes (B″) and (C″)

The process for preparing the inverse microlatex (B) of example 1 is carried out in the presence of a larger amount of water, so as to obtain an inverse microlatex (B″) that is in the form of opalescent to translucent oily compositions, containing 49% by weight of a mixture of oily phase and surfactants, 15% by weight of crosslinked sodium polyacrylate and 36% by weight of water.

The process for preparing the inverse microlatex (C) of example 1 is carried out in the presence of a larger amount of water, so as to obtain an inverse microlatex (C″) that is in the form of opalescent to translucent oily compositions, containing 49% by weight of a mixture of oily phase and surfactants, 15% by weight of crosslinked sodium polyacrylate and 36% by weight of water.

### Example 4: Preparation of Adjuvants According to the Invention

The polymeric oily adjuvants are prepared according to the following process:
a) preparing, with mechanical stirring and at ambient temperature, an oily phase comprising at least one oil and an emulsifying system comprising at least one water-in-oil surfactant (E₁) and/or oil-in-water surfactant (E₂);
b) adding the inverse microlatex or inverse latex, with moderate mechanical stirring (50 to 150 rpm) at ambient temperature;
c) maintaining the moderate mechanical stirring (50 to 150 rpm), at ambient temperature, until a homogeneous mixture is obtained.

For the purposes of the present invention, ambient temperature is understood to mean a temperature above or equal to 15° C. and below or equal to 30° C.

In this way, the ADJ1, ADJ2, ADJ3, ADJ′1 adjuvants are prepared and are characterized by the compositions described in table 1:

TABLE 1
adjuvants according to the invention and comparative
adjuvants (composition in %)

| | ADJ 1 | ADJ 2 | ADJ 3 | ADJ′1 |
|---|---|---|---|---|
| Marcol ™ 52 | 84% | 84% | 79.5% | 86% |
| Sorbitan oleate | 6.5% | 6.5% | 7.5% | 6.5% |
| Polysorbate 80 | 4.5% | 4.5% | 3% | 5.5% |
| Microlatex (F) (example 1) | 5% | 0% | 10% | 0% |
| Microlatex (C′) (example 2) | 0% | 5% | 0% | 0% |
| Inverse latex ⁽¹⁾ | 0% | 0% | 0% | 2% |

(1): Sodium polyacrylate that is in the form of an inverse latex, the preparation of which is described in patent FR2922767 B1.

### Example 5: Evaluation of the Adjuvants According to the Invention and of the Comparative Adjuvants

5.1 Filterability

The filterability of the adjuvants according to the invention and of the comparative adjuvants is evaluated according to the following protocol:
introduce 10 ml of adjuvant into a 2-piece 12 ml syringe,
connect a 0.22 μm PTFE syringe filter with a diameter of 25 mm,
apply a weight of 3310 g,
measure the mass filtered as a function of time.

The amount of adjuvant filtered is measured as a function of time and the results obtained for each adjuvant tested are recorded in the tables below:

TABLE 2
kinetics of the amount of filtered adjuvant ADJ1 according to the invention

| Time (s) | 10 | 20 | 30 | 60 | 90 | 120 |
|---|---|---|---|---|---|---|
| Mass | 0.48 | 1.12 | 1.68 | 3.27 | 4.85 | 6.34 |
| filtered (g) | | | | | | |

TABLE 3
kinetics of the amount of filtered adjuvant ADJ2 according to the invention

| Time (s) | 10 | 20 | 30 | 60 | 90 | 120 |
|---|---|---|---|---|---|---|
| Mass | 0.69 | 1.39 | 2.14 | 4.38 | 6.74 | 9.03 |
| filtered (g) | | | | | | |

TABLE 4
kinetics of the amount of filtered adjuvant ADJ3 according to the invention

| Time (s) | 10 | 20 | 30 | 60 | 90 | 120 |
|---|---|---|---|---|---|---|
| Mass | 0.43 | 0.85 | 1.28 | 2.56 | 3.83 | 5.11 |
| filtered (g) | | | | | | |

TABLE 5
kinetics of the amount of filtered adjuvant
ADJ′1 according to the invention

| Time (s) | 10 | 20 | 30 | 60 | 90 | 120 |
|---|---|---|---|---|---|---|
| Mass | 0 | 0 | 0 | 0 | 0 | 0 |
| filtered (g) | | | | | | |

The results recorded in tables 2 to 5 show that the filtration of the adjuvants according to the invention ADJ1, ADJ2, ADJ3 is faster than that of the comparative adjuvant ADJ′1 on a hydrophobic filter, in particular made of PTFE, with a mean pore diameter of 0.2 micrometers.

5.2 Study of the Stability of the Adjuvants According to the Invention and of the Comparative Adjuvants

The stability of the adjuvants according to the invention ADJ1, ADJ2, ADJ3 and of the comparative adjuvant ADJ′1 is evaluated according to the following protocol:

i) An amount of 90 ml of the composition to be tested, contained in a 100 ml flask, is introduced into a climatic chamber regulated at 20° C., for a period of one year. The visual appearance of the compound tested is evaluated before being placed in the stability test in the chamber and after a period of one month (M1), three months (M3), six months (M6) and one year (Y1).

ii) An amount of 90 ml of the composition to be tested, contained in a 100 ml flask, is introduced into a climatic chamber regulated at 37° C., for a period of one month (M1). The visual appearance of the composition tested is evaluated before being placed in the stability test in the chamber and after a period of one month.

Stability is understood to mean the absence of phase separation and/or observed sedimentation. The results of the observations are recorded in table 6 below.

TABLE 6
stability results of the adjuvants ADJ1, ADJ2, ADJ3 according
to the invention, and of the comparative adjuvant ADJ′1

| | | | |
|---|---|---|---|
| ADJ 1 | ADJ 2 | ADJ 3 | ADJ′1 |

Stability at 20° C.

| | | | | |
|---|---|---|---|---|
| 1 month (M1) | Clear | Clear | Clear | Heterogenous |
| 2 months (M3) | Clear | Clear | Clear | Heterogenous |
| 6 months (M6) | Clear | Clear | Clear | Heterogenous |
| 1 year (Y1) | Clear | Clear | Clear | Heterogenous |

Stability at 37° C.

| | | | | |
|---|---|---|---|---|
| 1 month (M1) | Clear | Clear | Clear | Heterogenous |

(Clear): homogeneous and clear, a single phase

(Heterogeneous): heterogeneous, two or three phases observed, presence of deposit.

The adjuvants ADJ1, ADJ2, ADJ3, ADJ′1 according to the invention have homogeneous appearances under the storage conditions described below, whereas heterogeneous appearances (phase separation and sedimentation) are observed over time and at various temperatures for the comparative adjuvant ADJ′1.

5.3. Characterization of the Stability Properties of Vaccine Compositions Containing Adjuvants According to the Invention

The stability properties of the placebo vaccine emulsions containing the adjuvants according to the invention ADJ1, ADJ2, ADJ3, are evaluated on an amount of 200 grams (prepared in a 250 ml low form beaker) according to the following protocol, using a Silverson L4 or L5 rotor-stator mixer fitted with its standard head provided with a standard emulsion screen:

i/ 60 grams of aqueous phase are added to 140 grams of adjuvant with mechanical stirring using a Silverson L4 or L5 mixer at a rotational speed of 1000 rpm (during this step the stirring head should be placed 0.5 cm from the bottom of the beaker);

ii/ the emulsion is produced by subjecting the mixture obtained in step i/ to high shear (with the Silverson L4 or L5 mixer) for a period of 3 minutes at a rotational speed of 4000 rpm (or 7 m/s).

The emulsions obtained at the end of step ii/ are fluid, homogeneous and injectable. The term “fluid” more particularly means a liquid emulsion, the dynamic viscosity of which, measured using a Brookfield LVDV1+ equipped with an M62 spindle at a rotational speed of 60 rpm, at 20° C., is between 30 and 40 mPa·s.

The stability of the emulsions obtained is characterized as follows:

i) An amount of 25 ml of the composition to be tested, contained in a 30 ml flask, is introduced into a climatic chamber regulated at 4° C., for a period of one year. The visual appearance of the compound tested is evaluated before being placed in the stability test in the chamber and after a period of one month (M1), three months (M3), six months (M6) and one year (Y1).

ii) An amount of 25 ml of the composition to be tested, contained in a 30 ml flask, is introduced into a climatic chamber regulated at 20° C., for a period of one year. The visual appearance of the compound tested is evaluated before being placed in the stability test in the chamber and after a period of one month (M1), three months (M3), six months (M6) and one year (A1).

iii) An amount of 25 ml of the composition to be tested, contained in a 30 ml flask, is introduced into a climatic chamber regulated at 37° C., for a period of one month (M1). The visual appearance of the composition tested is evaluated before being placed in the stability test in the chamber and after a period of one month.

Stability is understood to mean the absence of phase separation and/or observed sedimentation. The results of the observations are recorded in table 7 below.

TABLE 7
stability results of the emulsions containing the adjuvants
ADJ1, ADJ2, ADJ3 according to the invention

| | | |
|---|---|---|
| Emulsion | Emulsion | Emulsion |
| ADJ 1 | ADJ 2 | ADJ 3 |

Stability at 4° C.

| | | | |
|---|---|---|---|
| 1 month (M1) | H | H | H |
| 2 months (M3) | H | H | H |
| 6 months (M6) | H | H | H |
| 1 year (Y1) | H | H | H |

Stability at 20° C.

| | | | |
|---|---|---|---|
| 1 month (M1) | H | H | H |
| 2 months (M3) | H | H | H |
| 6 months (M6) | H | H | H |
| 1 year (Y1) | H | H | H |

Stability at 37° C.

| | | | |
|---|---|---|---|
| 1 month (M1) | H | H | H |

(H): homogeneous, only one phase observed

5.4 Characterization of the Immunological Properties of Vaccines Containing Adjuvants According to the Invention

The adjuvant properties of the polymeric oily adjuvants ADJ1, ADJ2 and ADJ3 according to the invention and as described in the preceding examples were characterized on several vaccine models and several animal species.

During a first test, the adjuvant according to the invention ADJ3 was formulated with a solution of ovalbumin to obtain a vaccine intended to be injected into mice.

In a second test, the adjuvant according to the invention ADJ2 was formulated with a bacterial antigen medium consisting of inactivated Pasteurella multocida bacteria to obtain a vaccine intended to be administered to avian species.

In a third test, the adjuvant according to the invention ADJ1 was used for the formulation of a viral vaccine intended for avian species against Newcastle disease and H9N2 influenza. These tests demonstrate the vaccine adjuvant properties of the adjuvants according to the invention in several species and several antigen models.

The results obtained are presented below.

5.4.1 Trial 1: Trial in Mice, with Ovalbumin as Antigen

The trial is carried out on OF1 mice in a 90-day vaccination protocol with ovalbumin (OVA) as an antigen model. The vaccines were prepared from an antigen solution of OVA prepared in physiological serum at 10 mg/ml, and sterilized by filtration on a 0.22 μm filter. The formulation of the vaccine comprising the OVA antigen and the ADJ3 adjuvant according to the invention is carried out by emulsification through an i-connector in an adjuvant according to the invention ADJ3/antigen medium ratio of 70/30 (volume/volume).

TABLE 8
composition of the vaccines tested

| | ADJ 3 | Physiological | OVA 10 mg/ml |
|---|---|---|---|
| Groups | μl | Serum μl | μl |
| Test Vaccine Group | 1400 | 580 | 20 |
| Antigen Control Vaccine Group | | 1980 | 20 |

The safety of the vaccines is assessed by observing local reactions at the injection sites. Vaccine efficacy is assessed by detection of IgG1 and IgG2a antibodies in the blood by the ELISA method. This detection is carried out on the day of vaccination (“primo-vaccination” on DO), then after 14 days (D14), at the time of the booster vaccination on the twenty-eighth day (D28), then on the forty-second day (D42), then on the fifty-sixth day (D56), then on the ninetieth day (D90) at the time of euthanasia.

No local reaction was observed among the members of the test group, thus signifying that the vaccine comprising the ADJ3 adjuvant according to the invention was well tolerated.

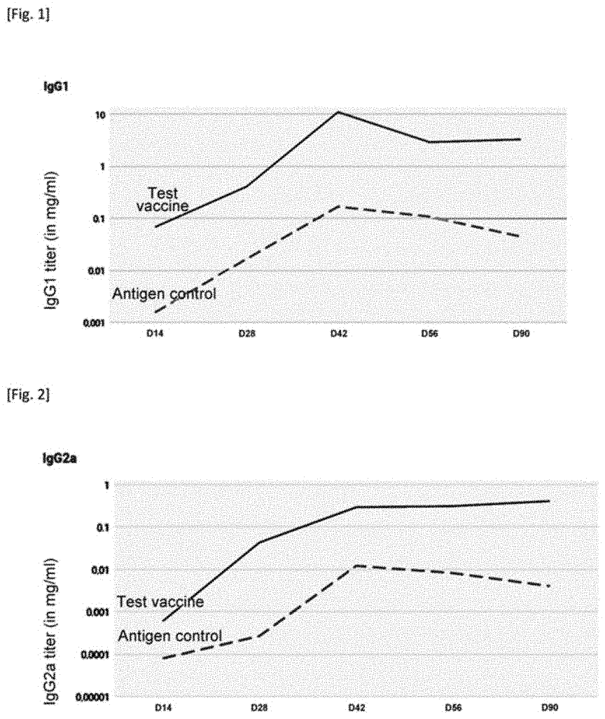

FIGS. 1 and 2 show the assay of the IgG1 and IgG2a antibodies on D14, D28, D42, D56 and D90.

FIG. 1 is a graph representing the response of the IgG1 antibody against the OVA antigen in mice for a vaccine comprising the ADJ3 adjuvant according to the invention.

FIG. 2 is a graph representing the response of the IgG2a antibody against the OVA antigen in mice for a vaccine comprising the ADJ3 adjuvant according to the invention.

Significantly higher antibody titers are observed for the vaccine comprising the ADJ3 adjuvant according to the invention compared to the non-adjuvanted vaccine comprising the antigen, for the two classes of antibodies, which confirms the vaccine adjuvant properties of the formula developed.

5.4.2 Trial 2: Vaccine Trial in Chickens, Pasteurella multocida Bacterial Antigen

This trial was carried out on chickens in a 42-day vaccination protocol against the Pasteurella multocida bacterial pathogen. The animals used in this experiment are red chickens aged 36 days at the time of vaccination (DO). Each vaccine dose contains 1 dose (0.5 ml)=0.5×10⁸ CFU or 1.10⁸ CFU/ml of inactivated Pasteurella multocida bacteria. The vaccine groups consist of 11 male and female chickens randomly distributed between the groups.

TABLE 9
composition of the vaccines tested

| | | Adjuvant | Antigen | Phys. |
|---|---|---|---|---|
| Group | Antigen dose | ADJ 2 (g) | (ml) | Serum (g) |
| Test Vaccine | 0.5 × 10⁸ UFC | 21 | 1.5 | 7.5 g |
| | 0.5 ml | | | |
| Antigen | 0.5 × 10⁸ UFC | | 1.5 | 28.5 g |
| Control | 0.5 ml | | | |

The formulation containing the ADJ2 adjuvant according to the invention is prepared using a Tube Drive emulsifier (sold by the company Ika) in sterile DT50 tubes in an ADJ2 adjuvant according to the invention/antigen medium ratio of 70/30 (weight/weight): speed 3 for 2 minutes (1100 rpm) for the pre-emulsion then speed 9 for 6 minutes (4000 rpm).

The animals are vaccinated on DO. Local reactions are observed at slaughter on D42. Blood samples will be taken on DO, D14, D42. Antigen-specific ELISA assays will be performed for the detection of antibody levels in serums using a commercial detection kit (ID Screen Pasteurella multocida chicken and turkey indirect kit sold by ID-VEt).

The vaccine comprising the ADJ2 adjuvant according to the invention is well tolerated in chickens, since no critical local reaction is observed at slaughter. Significantly higher antibody titers are also observed for the vaccine adjuvanted with the ADJ2 adjuvant compared with the non-adjuvanted vaccine in chickens, as shown in table 10 below.

TABLE 10
IgY antibody response against Pasteurella multocida in chickens for
a vaccine comprising the adjuvant ADJ2 according to the invention.

| | D0 | D14 | D42 |
|---|---|---|---|
| Test Vaccine Group | 213 | 7506 | 6176 |
| IgY titer (in AU) | | | |
| Antigen Control | 225 | 277 | 791 |
| Vaccine Group | | | |
| IgY titer (in AU) | | | |

5.4.3 Trial 3: Vaccine Trial in Chickens, Newcastle Disease/Avian Influenza Viral Antigen

This trial was carried out on chickens in a 28-day vaccination protocol with a bivalent inactivated viral vaccine against Newcastle disease LaSota strain (NDV) and H9N2 avian influenza (AIV). The animals used in this experiment are SPF (specific pathogen free) chickens aged 28 days at the start of the experiment (DO). The vaccine groups are formed as follows in table 11:

TABLE 11
composition of the vaccine groups tested

| Groups | No. of animals | AI-ND vaccine dose | Valency tested |
|---|---|---|---|
| Test vaccine | 10 | 0.3 ml | AIV |
| | 10 | 0.02 ml | NDV |
| Unvaccinated | 5 | / | AIV |
| control group | 5 | / | NDV |

The test vaccine is formulated using the polymeric oily adjuvant ADJ1 according to the invention by emulsification in an ADJ1 adjuvant according to the invention/antigen medium ratio of 70/30 (weight/weight) with the antigen medium containing the two AIV and NDV inactivated viral valencies. The control group is not vaccinated.

The vaccines are injected intramuscularly on DO. Blood samples are taken on D0, D7, D14, D21 and D28 after vaccination and analyzed by hemagglutination inhibition test to determine the specific antibody titers against each valency (AIV and NDV). In the AIV group, a protection challenge is carried out on D28 after vaccination to measure the viral load after challenge (2.10⁶ EID₅₀ in 0.2 ml intravenously of XZ strain H9N2 AIV virus). Oropharyngeal and cloacal swabs are collected 5 days after viral challenge and inoculated into SPF chicken embryos to measure viral presence after two generations of transmission.

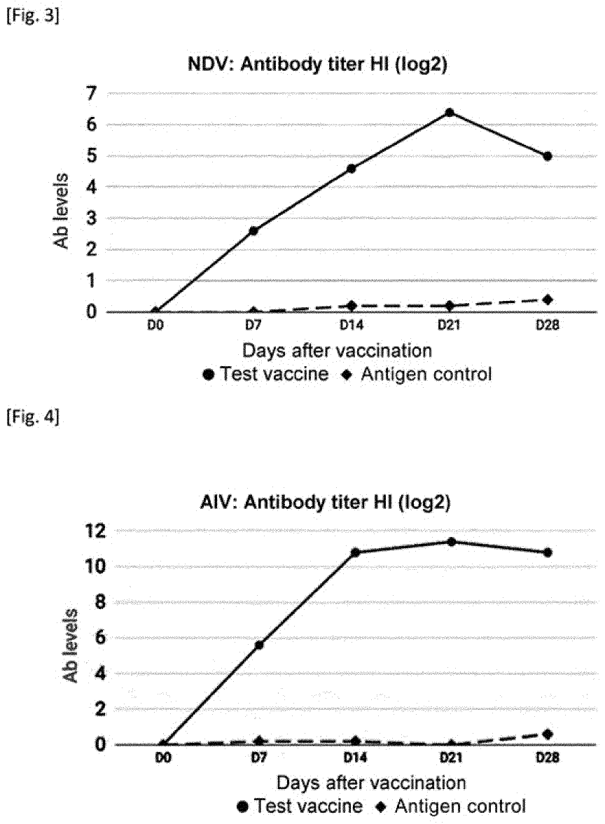

Strong antibody titers are observed in the vaccinated group after vaccination for the two valencies (FIG. 1 and FIG. 2). After virulent challenge, no mortality was observed, and in the vaccinated group there was also no viral load observed in the swab samples, which demonstrates complete protection against the AIV viral challenge (FIG. 3).

FIG. 3 is a graph representing the change in the titre of antibodies against Newcastle disease LaSota strain (NDV) from DO to D28.

FIG. 4 is a graph representing the change in the titre of antibodies against H9N2 avian influenza (AIV) from DO to D28.

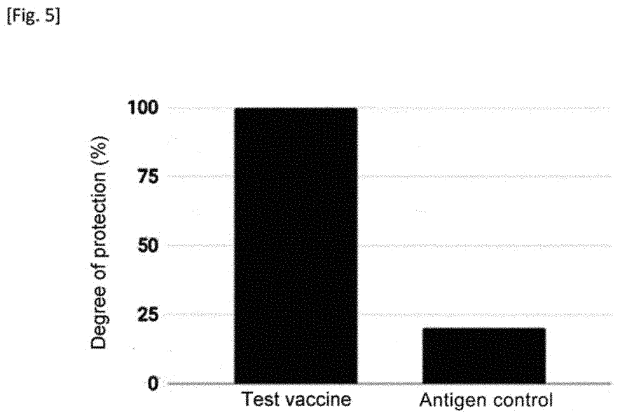

FIG. 5 is a graph representing the degree of protection (number of animals protected/total number) obtained by the test vaccine group in comparison with the control group.

5.5 Experimental Conclusion

The adjuvants according to the invention are characterized by:
filtration kinetics, in particular on hydrophobic filters (in particular made of polytetrafluoroethylene (PTFE)) with a mean pore diameter of 0.2 micrometers, suitable for obtaining a sterile adjuvant,
stability over time at 20° C. and 37° C., i.e. retaining a homogeneous and clear appearance, without displaying phase separation and/or sedimentation phenomena,
obtaining stable vaccine emulsions, formed by emulsification of said adjuvants in the presence of vaccine aqueous phase,
an adjuvant effect of immunity in various animal species in vaccine compositions in the presence of various antigen media.

1. A vaccine adjuvant comprising at least, as inverse microlatex, an inverse microemulsion comprising at least one polyelectrolyte-type polymer.

2. The vaccine adjuvant as claimed in claim 1, wherein the inverse microlatex comprises an oily phase, an aqueous phase, at least one water-in-oil (W/O) surfactant, at least one oil-in-water (O/W) surfactant and an anionic and crosslinked polyelectrolyte; with said anionic and crosslinked polyelectrolyte comprising at least one crosslinking monomer and at least one hydrophilic monomer unit.

3. The vaccine adjuvant as claimed in claim 2, wherein the monomer unit originates from acrylic acid that is completely or partially salified with an alkali or alkaline-earth metal salt or an ammonium salt.

4. The vaccine adjuvant as claimed in claim 3, wherein the acrylic acid is totally or partially salified with a sodium salt or an ammonium salt.

5. The vaccine adjuvant as claimed in claim 2, wherein the anionic and crosslinked polyelectrolyte comprises a monomer unit of formula (1): 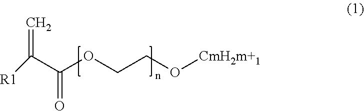
with:
R1 chosen from —H, —CH₃, —C₂H₅ and —C₃H₇,
n between 0 and 50, and
m between 8 and 22.

6. The vaccine adjuvant as claimed in claim 1, wherein said adjuvant further comprises an oil (H₁), at least one water-in-oil surfactant (E₁) and at least one oil-in-water surfactant (E₂).

7. The vaccine adjuvant as claimed in claim 6, further comprising between 1% and 10% by weight of water-in-oil surfactant (E₁).

8. The vaccine adjuvant as claimed in claim 6, further comprising between 1% and 10% by weight of water-in-oil surfactant (E₂).

9. The vaccine adjuvant as claimed in claim 6, comprising, per 100% of its weight:
a) from 50% to 97.5% by weight of said oil (H₁);
b) from 1% to 10% by weight of said water-in-oil surfactant (E₁);
c) from 1% to 10% by weight of said oil-in-water surfactant (E₂); and
d) from 0.5% to 30% by weight of at least one inverse microlatex;
it being understood that the sum of the weight contents a)+b)+c)+d) is equal to 100%.

10. The vaccine adjuvant as claimed in claim 6, wherein the oil (H₁) is a white mineral oil.

11. (canceled)

12. A vaccine comprising the adjuvant as defined in claim 1 and also at least one aqueous solution (S) of at least one antigen or of at least one in vivo generator of a compound comprising an amino acid sequence.

13. A vaccine comprising the adjuvant as defined in claim 1 and also at least one aqueous solution (S) of at least one antigen or of at least one in vivo generator of a compound comprising an amino acid sequence, wherein the vaccine comprises:
from 10% to 80% by weight of the adjuvant, and,
from 20% to 90% by weight of the aqueous solution (S).

14. The vaccine as claimed in claim 12, wherein said vaccine is in the form of a water-in-oil emulsion or an oil-in-water emulsion.

15. A process for preparing a vaccine adjuvant as defined in claim 1, comprising a step of sterilizing the vaccine adjuvant by filtration or by autoclaving.

16. The process as claimed in claim 15, wherein said process comprises the following steps:
a) preparing, with mechanical stirring and at ambient temperature, an oily phase comprising at least one oil and an emulsifying system comprising at least one water-in-oil surfactant (E₁) and/or oil-in-water surfactant (E₂);
b) adding the at least one inverse microlatex with mechanical stirring at ambient temperature;
c) maintaining the mechanical stirring, at ambient temperature, until a homogeneous mixture is obtained.

17. A process for preparing a vaccine, comprising the following steps:
a) preparing the vaccine adjuvant as claimed in the process as defined in claim 15, and
b) mixing the vaccine adjuvant obtained in step a) with an antigen medium.

18. The vaccine of claim 5, wherein R1 is CH₃.

19. The vaccine adjuvant of claim 7, comprising from 3% to 8% by weight of water-in-oil surfactant.

20. The vaccine adjuvant as claimed in claim 6, comprising, per 100% of its weight:
a) from 60% to 90% by weight of said oil (H₁);
b) from 3% to 8% by weight of said water-in-oil surfactant (E₁);
c) from 3% to 8% by weight of said oil-in-water surfactant (E₂); and
d) from 1% to 10% by weight of at least one inverse microlatex;
it being understood that the sum of the weight contents a)+b)+c)+d) is equal to 100%.

21. The vaccine adjuvant as claimed in claim 3, wherein the anionic and crosslinked polyelectrolyte comprises a monomer unit of formula (1): 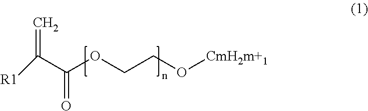
with:
R1 chosen from —H, —CH₃, —C₂H₅ and —C₃H₇,
n between 0 and 50, and
m between 8 and 22.
